(19) United States
(12) Patent Application Publication
(10) Pub. No.: US 2006/0110353 A1
(43) Pub. Date: May 25, 2006

(54) Aerosol device comprising at least one high molecular weight polyurethane

(76) Inventors: Isabelle Rollat-Corvol, Paris (FR); Pascale Cothias, Montigny le Bretonneu (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/260,439

(22) Filed: Oct. 28, 2005

Related U.S. Application Data
(60) Provisional application No. 60/646,486, filed on Jan. 25, 2005.

(30) Foreign Application Priority Data
Oct. 28, 2004 (FR) 04 52475

(51) Int. Cl. A61K 8/87

(52) U.S. Cl. 424/70.17

(57) ABSTRACT

Disclosed herein is a composition for cosmetic treatment of keratin materials, packaged in an aerosol device comprising (a) at least one propellant and (b) a hairstyling composition comprising at least one polyurethane having a number-average molecular weight ranging from 400 000 to 5 000 000 g/mol, in a cosmetically acceptable medium comprising water. Also disclosed herein is an aerosol device comprising the composition disclosed herein.

This application claims benefit of U.S. Provisional Application No. 60/646,486, filed Jan. 25, 2005, the contents of which are incorporated herein by reference. This application also claims benefit of priority under 35 U.S.C. § 119 to French Patent Application No. 04 52475, filed Oct. 28, 2004, the contents of which are also incorporated by reference.

Disclosed herein is a composition for cosmetic treatment of keratin materials, packaged in an aerosol device comprising (a) at least one propellant and (b) a hairstyling composition comprising at least one high molecular weight polyurethane in a cosmetically acceptable medium comprising water. Also disclosed herein is an aerosol device comprising at least one propellant and at least one high molecular weight polyurethane, and also to a method of styling the hair using such a device.

As used herein, a “high molecular weight polyurethane” is a polyurethane having a number-average molecular weight ranging from 400 000 to 5 000 000.

Hairstyling compositions packaged in the form of an aerosol spray generally contain a large alcohol fraction. Now, cosmetic products with a high alcohol content are subject to specific monitoring, especially in the United States, following the recent sensitization of public opinion to the ecological problems resulting from the emission of volatile organic products into the atmosphere.

One solution for decreasing the amount of alcohol is replacement with an equivalent amount of water. This introduction of large amounts of water into aerosol sprays intended for fixing the hair, such as lacquers, results, however, in a considerable unwanted deformation of the hair and in degradation of the cosmetic properties. Moreover, most propellants of hydrocarbon type are incompatible with water and their use in compositions with a high water content is, as a result, generally impossible. Once applied to the hair, the fixative compounds should allow the hair to be fixed.

The polymers presently in use as fixatives in styling compositions do not allow the shape of the hairstyle to be retained when it comes into contact with water for a prolonged period, such as, for example, when contacted with rain, perspiration, or during bathing: bathing in the sea or in a swimming pool.

Surprisingly, the inventors have discovered that the use of a cosmetic treatment composition packaged in an aerosol device comprising (a) at least one propellant and (b) a hairstyling composition comprising at least one polyurethane having a molecular number-average molecular weight ranging from 400 000 to 5 000 000, in a cosmetically acceptable medium comprising water, allows hairstyles to be fixed and shaped and also allows the shape of the hairstyles to be retained when they come into contact with liquid water for a prolonged period.

As used herein, this phenomenon is referred to as “water resistance”.

As used herein, “prolonged period” means contact with water for a minimum period of one minute, for example, 10 minutes and further, for example, 20 minutes.

The compositions disclosed herein also may make it possible to obtain a hairstyle resistant to atmospheric humidity.

The compositions disclosed herein may allow effective fixative and effective holding of the hair, in other words a styling effect that lasts throughout the day, or even for several days, which exhibits good water resistance, for example, resistance to repeated bathing, wherein these compositions may also be removed by shampooing.

The compositions disclosed herein also may make it possible to impart good cosmetic properties.

The present disclosure therefore provides a cosmetic treatment composition packaged in an aerosol device comprising (a) at least one propellant and (b) a hairstyling composition comprising at least one polyurethane having a number-average molecular weight ranging from 400 000 to 5 000 000, in a cosmetically acceptable medium comprising water.

Also disclosed herein is a method for styling the hair comprising spraying the mixture comprised in an aerosol device comprising a composition for cosmetic treatment of keratin materials, packaged in an aerosol device comprising (a) at least one propellant and (b) a hairstyling composition comprising at least one polyurethane having a number-average molecular weight ranging from 400 000 to 5 000 000 g/mol, in a cosmetically acceptable medium comprising water over the hair that has been shaped and in allowing the hair thus treated to dry.

Also disclosed herein is a process for treating the hair comprising spraying to said hair a mixture comprised in an aerosol device comprising a composition for cosmetic treatment of keratin materials, packaged in an aerosol device comprising (a) at least one propellant and (b) a hairstyling composition comprising at least one polyurethane having a number-average molecular weight ranging from 400 000 to 5 000 000 g/mol, in a cosmetically acceptable medium comprising water wherein the hair is fixed and shaped and retains the shape of hairstyles when said hair comes into contact with water for a prolonged period. According to this embodiment, the process for treating the hair comprising spraying the hair with a mixture comprised in an aerosol device comprising the composition as disclosed herein may make it possible to obtain a hairstyle (shaping of the hair) resistant to water.

Hairstyling Composition

As used herein, “polyurethane” means polycondensates comprising at least one polyurethane sequence. They are described, for example, in European Patent Nos. EP 0 751 162, EP 0 637 600, EP 0 656 021, EP 0 619 111 and EP 0 648 485, French Patent No. FR 2 743 297, and in PCT Publication No. WO 94/03510.

The polyurethanes disclosed herein may be soluble in the cosmetically acceptable aqueous medium, for example, after neutralization with an organic or inorganic base, or else may form a dispersion in said medium. In that case the dispersion may comprise at least 0.05% of surfactant, allowing the non-associative polyurethane to be dispersed and to be held in dispersion.

In one embodiment, the dispersion described herein may comprise a surfactant, for example, a nonionic surfactant. The average size of the particles of the non-associative polyurethane in the dispersion may range from 0.1 to 1 micrometer.

For example, the polyurethane may be formed by an arrangement of blocks, this arrangement being obtained, for example, from:
(1) at least one compound comprising at least two active hydrogen atoms per molecule;
(2) at least one diol or a mixture of diols comprising acidic functions, or their salts; and
(3) at least one di- or polyisocyanate.

The at least one compound (1) may be chosen from diols, diamines, polyesterols, polyetherols and mixtures thereof.

The at least one compound (1) may be, for example, linear polyethylene glycols and polypropylene glycols, for example, those obtained by reacting ethylene oxide or propylene oxide with water or reacting diethylene or dipropylene glycol in the presence of sodium hydroxide as catalyst. These polyalkylene glycols generally have a molecular mass ranging from 600 to 20 000.

Other organic compounds which may be use, for example, are those having mercapto, amino, carboxyl or hydroxyl groups. These may include polyhydroxy compounds such as polyether-diols, polyester-diols, polyacetal-diols, polyamide-diols, polyester-polyamide-diols, poly(alkylene ether)-diols, polythioether-diols and polycarbonate-diols.

Polyether-diols are, for example, condensation products of ethylene oxide, propylene oxide or tetrahydrofuran, their copolymerization or condensation products, grafted or in blocks, such as mixtures of condensates of ethylene oxide and propylene oxide, and the products of high-pressure polymerization of olefins with alkylene oxide condensates. Appropriate polyether-diols are prepared for example by condensing alkylene oxides and polyhydric alcohols, such as ethylene glycol, propylene 1,2-glycol and 1,4-butanediol.

The polyester-diols, polyester-amides and polyamide-diols may be, for example, saturated and are obtained, for example, from the reaction of saturated or unsaturated polycarboxylic acids with polyhydric alcohols, diamines or polyamines. These compounds may be prepared using, for example, adipic acid, succinic acid, phthalic acid, terephthalic acid and maleic acid. Polyhydric alcohols appropriate for preparing the polyesters include, for example, ethylene glycol, propylene 1,2-glycol, 1,4-butanediol, neopentyl glycol and hexanediol. It is also possible to use amino alcohols, for example ethanolamine. Appropriate diamines for preparing the amide-polyesters are ethylenediamine and hexamethylenediamine.

Appropriate polyacetals may be prepared, for example, from 1,4-butanediol or hexanediol and formaldehyde. Appropriate polythioethers may be prepared, for example, by a condensation reaction of thioglycols, alone or in combination with other glycols such as ethylene glycol, propylene 1,2-glycol or with other polyhydroxy compounds. Polyhydroxy compounds already comprising urethane groups, natural polyols, which may additionally be modified, for example castor oil and carbohydrates, may also be used.

For example, the at least one compound (1) may be a polyesterol, for example, a polyester-diol formed by the reaction of at least one (di)-polyol (1ₐ) and at least one acid (1_{b}). The (di)-polyol (1ₐ) may be chosen from neopentyl glycol, 1,4-butanediol, hexanediol, ethylene glycol, diethylene glycol, propylene glycol, butylene glycol, neopentyl glycol and (di)-polyethylene glycol. The acid (1_{b}) may be chosen from phthalic acid, isophthalic acid, adipic acid and (poly)lactic acid.

For example, the at least one compound (2) may be a hydroxy carboxylic acid such as dimethylolpropanoic acid (DMPA) or a 2,2-hydroxymethyl-carboxylic acid. For example, the at least one compound (2) is useful as a coupling block. The at least one compound (2) may include at least one poly(α,α-dihydroxy carboxylic acid).

The at least one compound (2) may be chosen from 2,2-di(hydroxymethyl)acetic acid, 2,2-dihydroxymethylpropionic acid, 2,2-dihydroxymethylbutyric acid and 2,2-dihydroxymethylpentanoic acid.

The at least one compound (3) may be selected, for example, from hexamethylene diisocyanate, isophorone diisocyanate (IPDI), tolylene diisocyanate, diphenylmethane 4,4′-diisocyanate (DPMD) and dicyclohexylmethane 4,4′-diisocyanate (DCMD), methylenedi-p-phenyl diisocyanate, methylenebis(4-cyclohexyl isocyanate), toluene diisocyanates, naphthalene 1,5-diisocyanate, diphenylmethane 4,4′-diisocyanate, 2,2′-dimethyldiphenylmethane 4,4′-diisocyanate, phenylene 1,3-diisocyanate, phenylene 1,4-diisocyanate, mixtures of toluene 2,4- and 2,6-diisocyanates, 2,2′-dichloro-4,4′-diisocyanatodiphenylmethane, 2,4-dibromo-1,5-diisocyanatonaphthalene, butane 1,4-diisocyanate, hexane 1,6-diisocyanate and cyclohexane 1,4-diisocyanate.

The polyurethane may be formed with the aid of at least one additional compound (4) serving generally to extend its chain. The at least one compound (4) may be selected, for example, from saturated or unsaturated glycols such as ethylene glycol, diethylene glycol, neopentyl glycol, and triethylene glycol; amino alcohols such as ethanolamine, propanolamine and butanolamine; heterocyclic, aromatic, cycloaliphatic and aliphatic primary amines; diamines; carboxylic acids such as aliphatic, aromatic and heterocyclic carboxylic acids, for instance oxalic, succinic, glutaric, adipic, sebacic and terephthalic acids; and aminocarboxylic acids. For example, the at least one compounds (4) may be aliphatic diols.

The polyurethanes may, for example, be formed from at least one additional compound (5) having a silicone skeleton, such as polysiloxanes, polyalkylsiloxanes or polyarylsiloxanes, for example, polyethylsiloxanes, polymethylsiloxanes and polyphenylsiloxanes, optionally comprising hydrocarbon chains grafted on the silicon atoms.

The polyurethanes used may, for example, comprise a base repeating unit conforming to the general formula (I):
—O—B—O—CO—NH—R—NH—CO— (I)
wherein:

B is chosen from C₁ to C₃₀ divalent hydrocarbon groups, which are unsubstituted or substituted by a group chosen from at least one carboxylic acid function and at least one sulfonic acid function, wherein the at least one carboxylic acid function and the at least one sulfonic acid function are in free form or are fully or partially neutralized by an inorganic or organic base, and

R is chosen from divalent groups chosen from C₁ to C₂₀ aliphatic hydrocarbon groups; C₃ to C₂₀ cycloaliphatic hydrocarbon groups; and C₆ to C₂₀ aromatic hydrocarbon groups; such as, for example, C₁ to C₂₀ alkylene groups, C₆ to C₂₀ arylene groups or C₃ to C₂₀ cycloalkylene groups, or combinations thereof; wherein each group is substituted or unsubstituted.

The group R may be chosen from: 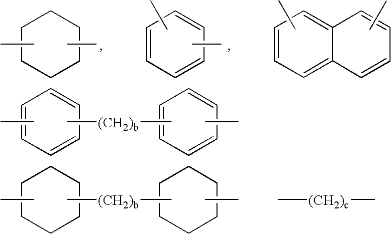
wherein:
b is chosen from an integer ranging from 0 to 3, and
c is chosen from an integer ranging from 1 to 20, for example, from 2 to 12.

For example, the group R may be chosen from hexamethylene, 4,4′-diphenylenemethane, 2,4- and/or 2,6-tolylene, 1,5-naphthylene, p-phenylene and methylene-4,4-biscyclohexyl groups and the divalent group derived from isophorone.

In one embodiment, the polyurethane disclosed herein may further comprise at least one polysiloxane sequence.

The polyurethanes disclosed herein may be, for example, non-associative, meaning that their structure does not include an alkyl or alkenyl chain comprising more than 10 carbon atoms.

In one embodiment, the polyurethanes may be present in an amount ranging from 0.05% to 40% by weight, for example, from 0.1% to 20% by weight and further, by example, from 1% to 8% by weight, relative to the total weight of the composition.

In another embodiment, the polyurethane may have a molecular mass ranging from 400 000 to 3 000 000, for example, from 1 000 000 to 2 500 000.

In another embodiment, the polyurethane may be, for example, chosen from Avalure UR-450, sold by Noveon, which is an anionic copolymer formed from PPG-1 7 (polypropylene glycol with a number of units, n, of 17)/IPDI (isophorone diisocyanate)/DMPA (dimethylolpropionic acid). In an aqueous medium it is in the form of a dispersion. Its molecular weight is 1 830 000 g/mol.

The cosmetically acceptable aqueous medium may consist only of water, or of a mixture of water and at least one cosmetically acceptable solvents such as a C₁-C₄ lower alcohol, for instance ethanol, isopropanol, tert-butanol, n-butanol; alkylene polyols, for instance propylene glycol; polyol ethers; and mixtures thereof. The additional solvent may be, for example, ethanol.

Although the hairstyling compositions comprised in the aerosol device disclosed herein may be free of lower alcohols, they may, for example, comprise up to 70% by weight, relative to the total weight of the mixture comprised in the aerosol device, of at least one lower alcohols such as ethanol or isopropanol. This content may be, for example, less than 50%.

In the hairstyling compositions comprised in the aerosol device, water is present in an amount ranging from 5 to 95% by weight, for example, between 20 to 90%, relative to the total weight of the mixture comprised in the aerosol device.

Propellant

The propellant may be, for example, any liquefiable gas normally used in aerosol devices, such as dimethyl ether, C₃₋₅ alkanes, 1,1-difluoroethane, mixtures of dimethyl ether and of C₃₋₅ alkanes, and mixtures of 1,1-difluoroethane and of dimethyl ether and/or of C₃₋₅ alkanes. C₃₋₅ alkanes, for example, propane, n-butane and isobutane, may be used as propellant. The propellant may be present in an amount ranging from 1 to 90% of the total weight of the mixture (propellant (a)+hairstyling composition (b)) comprised in the aerosol device, for example, ranging from 2 to 80%, and further, for example, from 3 to 70%.

In one embodiment, the hairstyling composition disclosed herein may also comprise at least one additive chosen from nonionic, anionic, cationic and amphoteric surfactants, nonionic, anionic, cationic and amphoteric additional polymers other than the fixative polymers used in the compositions disclosed herein, ceramides and pseudoceramides, vitamins and pro-vitamins, including panthenol, silicone or non-silicone water-soluble and liposoluble sunscreens, fillers and solid particles, for instance colored or uncolored mineral and organic pigments, nacreous agents and opacifiers, flakes, active particles, dyes, sequestering agents, plasticizers, solubilizers, acidifying agents, basifying agents, neutralizers, mineral and organic thickeners, antioxidants, hydroxy acids, penetrants, fragrances and preserving agents.

Other Fixative Polymers

All of the anionic, cationic, amphoteric and nonionic fixative polymers and mixtures thereof that are used in the art may be used in the compositions disclosed herein.

The fixative polymers may be soluble in the cosmetically acceptable medium or insoluble in said medium and in that case may be used in the form of dispersions of solid or liquid polymer particles (latex or pseudolatex).

The anionic fixative polymers generally used are polymers comprising groups derived from carboxylic, sulphonic or phosphoric acid, and have a number-average molecular mass ranging from 500 to 5 000 000.

The carboxylic groups are provided by unsaturated mono- or dicarboxylic acid monomers such as those conforming to the formula: 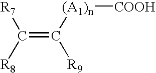
wherein:
n is an integer ranging from 0 to 10,
A₁ is chosen from methylene groups optionally joined to the carbon atom of the unsaturated group or to the adjacent methylene group when n is greater than 1, via a heteroatom such as oxygen or sulphur,
R₇ is chosen from hydrogen atoms, phenyl groups and benzyl groups,
R₈ is chosen from hydrogen atoms, lower alkyl groups and carboxyl groups, and
R₉ is chosen from hydrogen atoms, lower alkyl groups, —CH₂—COOH, phenyl groups and benzyl groups.

In the formula mentioned above, a lower alkyl group means a group ranging from 1 to 4 carbon atoms, for example, methyl and ethyl groups.

The anionic fixative polymers comprising carboxylic groups that may be used, for example, are:

A) acrylic or methacrylic acid homo- or copolymers or salts thereof, for example, the products sold under the names Versicol® E or K by Allied Colloid and Ultrahold® by BASF, the copolymers of acrylic acid and of acrylamide sold in the form of their sodium salts under the names Reten 421, 423 or 425 by Hercules, the sodium salts of polyhydroxycarboxylic acids;

B) copolymers of acrylic or methacrylic acid with a monoethylenic monomer such as ethylene, styrene, vinyl esters, acrylic or methacrylic acid esters, optionally grafted onto a polyalkylene glycol such as polyethylene glycol and optionally crosslinked. Such polymers are described, for example, in French Patent No. FR 1 222 944 and German Patent Application No. DE 2 330 956, the copolymers of this type comprising an optionally N-alkylated and/or hydroxyalkylated acrylamide unit in their chain as described, for example, in Luxembourg Patent Applications Nos. LU 75370 and 75371 or sold under the name Quadramer by American Cyanamid. Non-limiting mention may also be made of copolymers of acrylic acid and of C₁-C₄ alkyl methacrylate and terpolymers of vinylpyrrolidone, of acrylic acid and of methacrylate of C₁-C₂₀ alkyl, for example of lauryl, such as the product sold by ISP under the name Acrylidone® LM and methacrylic acid/ethyl acrylate/tert-butyl acrylate terpolymers such as the product sold under the name Luvimer® 100 P by BASF;

Non-limiting mention may also be made of methacrylic acid/acrylic acid/ethyl acrylate/methyl methacrylate copolymers as an aqueous dispersion, sold under the name Amerhold® DR 25 by Amerchol;

C) crotonic acid copolymers, such as those comprising vinyl acetate or propionate units in their chain and optionally other monomers such as allylic esters or methallylic esters, vinyl ether or vinyl ester of a linear or branched saturated carboxylic acid with a long hydrocarbon chain such as those comprising at least 5 carbon atoms, it being possible for these polymers optionally to be grafted or crosslinked, or alternatively another vinyl, allylic or methallylic ester monomer of an α- or β-cyclic carboxylic acid. Such polymers are described, for example, in French Patent Nos. 1 222 944, 1 580 545, 2 265 782, 2 265 781, 1 564 110 and 2 439 798. Commercial products falling into this class are the resins 28-29-30, 26-13-14 and 28-13-10 sold by National Starch;

D) copolymers of C₄-C₈ monounsaturated carboxylic acids or anhydrides chosen from:
copolymers comprising (i) at least one maleic, fumaric or itaconic acids or anhydrides and (ii) at least one monomer chosen from vinyl esters, vinyl ethers, vinyl halides, phenylvinyl derivatives, acrylic acid and its esters, the anhydride functions of these copolymers optionally being monoesterified or monoamidated. Such polymers are described, for example, in U.S. Pat. Nos. 2,047,398, and 2,723,248 and UK Patent No. GB 839 805. Commercial products are, for example, those sold under the names Gantreze AN or ES by ISP;
copolymers comprising (i) at least one maleic, citraconic or itaconic anhydride unit and (ii) at least one monomer chosen from allylic and methallylic esters optionally comprising at least one acrylamide, methacrylamide, α-olefin, acrylic or methacrylic ester, acrylic or methacrylic acid or vinylpyrrolidone group in their chain,
the anhydride functions of these copolymers optionally being monoesterified or monoamidated.

These polymers are described, for example, in French Patent Nos. FR 2 350 384 and 2 357 241;

E) polyacrylamides comprising carboxylate groups.

The homopolymers and copolymers comprising sulphonic groups are polymers comprising vinylsulphonic, styrenesulphonic, naphthalenesulphonic or acrylamidoalkylsulphon ic units.

These polymers may be chosen from:
polyvinylsulphonic acid salts having a molecular mass ranging from 1000 to 100 000, and also the copolymers with an unsaturated comonomer such as acrylic or methacrylic acids and their esters, and also acrylamide or its derivatives, vinyl ethers and vinylpyrrolidone;
polystyrenesulphonic acid salts such as the sodium salts that are sold for example under the names Flexan® 500 and Flexan® 130 by National Starch. These compounds are described in French Patent No. FR 2 198 719;
polyacrylamidesulphonic acid salts, such as those mentioned in U.S. Pat. No. 4,128,631, for example, polyacrylamidoethylpropanesulphonic acid sold under the name Cosmedia Polymer HSP 1180 by Henkel.

As another anionic fixative polymer that can be used in the composition disclosed herein, non-limiting mention may be made of the branched block anionic polymer sold under the name Fixate G-100 by Noveon.

In one embodiment, the anionic fixative polymers may be chosen from acrylic acid copolymers, such as the acrylic acid/ethyl acrylate/N-tert-butylacrylamide terpolymers sold, for example, under the name Ultrahold® Strong by BASF, copolymers derived from crotonic acid, such as vinyl acetate/vinyl tert-butylbenzoate/crotonic acid terpolymers and the crotonic acid/vinyl acetate/vinyl neododecanoate terpolymers sold, for example, under the name Résine 28-29-30 by National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez® by ISP, the copolymers of methacrylic acid and of methyl methacrylate sold under the name Eudragit® L by Rohm Pharma, the copolymers of methacrylic acid and of ethyl acrylate sold under the name Luvimer® MAEX or MAE by BASF, the vinyl acetate/crotonic acid copolymers sold under the name Luviset CA 66 by BASF, the vinyl acetate/crotonic acid copolymers grafted with polyethylene glycol sold under the name Aristoflex® A by BASF, and the polymer sold under the name Fixate G-100 by Noveon.

For example, the anionic fixative polymers may be chosen from methyl vinyl ether/monoesterified maleic anhydride copolymers sold under the name Gantrez® ES 425 by ISP, the acrylic acid/ethyl acrylate/N-tert-butylacrylamide terpolymers sold under the name Ultrahold® Strong by BASF, the copolymers of methacrylic acid and of methyl methacrylate sold under the name Eudragit® L by Rohm Pharma, the vinyl acetate/vinyl tert-butylbenzoate/crotonic acid terpolymers and the crotonic acid/vinyl acetate/vinyl neododecanoate terpolymers sold under the name Resine 28-29-30 by National Starch, the copolymers of methacrylic acid and of ethyl acrylate sold under the name Luvimer® MAEX or MAE by BASF, the vinylpyrrolidone/acrylic acid/lauryl methacrylate terpolymers sold under the name Acrylidone® LM by ISP, and the polymer sold under the name Fixate G-100 by Noveon.

The cationic fixative film-forming polymers may be chosen from polymers comprising primary, secondary, tertiary and/or quaternary amine groups forming part of the polymer chain or directly attached thereto, and having a molecular weight ranging from 500 to 5 000 000, for example, from 1000 to 3 000 000.

The cationic fixative film-forming polymers may be chosen from:

(1) homopolymers or copolymers derived from acrylic or methacrylic esters or amides and comprising at least one of the units of the following formulae: 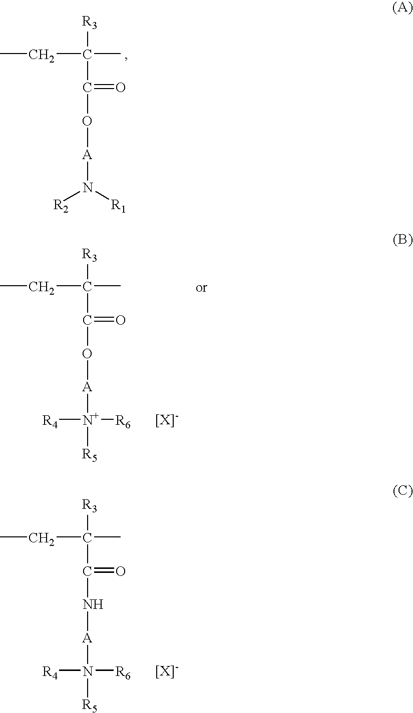
wherein:

R₃ is chosen from hydrogen atoms and CH₃ radicals;

A is chosen from linear or branched alkyl groups comprising from 1 to 6 carbon atoms and hydroxyalkyl groups comprising from 1 to 4 carbon atoms;

R₄, R₅ and R₆, which may be identical or different, are each chosen from alkyl groups comprising from 1 to 18 carbon atoms and benzyl radicals;

R₁ and R₂, which may be identical or different, are each chosen from hydrogen atoms and alkyl groups comprising from 1 to 6 carbon atoms;

X is chosen from methosulphate anions and halides, such as chloride or bromide.

The copolymers of class (1) also comprise at least one comonomer units that may be chosen from the class of acrylamides, methacrylamides, diacetone acrylamides, acrylamides and methacrylamides substituted on the nitrogen by lower (C₁-C₄) alkyl groups, groups derived from acrylic or methacrylic acids or esters thereof, vinyllactams such as vinylpyrrolidone or vinylcaprolactam, and vinyl esters.

Thus, among these copolymers of class (1), non-limiting mention may be made of:

copolymers of acrylamide and of dimethylaminoethyl methacrylate quaternized with dimethyl sulphate or with a dimethyl halide, such as that sold under the name Hercofloc® by Hercules,

copolymers of acrylamide and of methacryloyloxyethyltrimethylammonium chloride, described, for example, in European Patent Application No. EP-A-080 976 and sold under the name Bina Quat P 100 by Ciba Geigy,

copolymer of acrylamide and of methacryloyloxyethyltrimethylammonium methosulphate, such as that sold under the name Reten by Hercules,

quaternized or non-quaternized vinylpyrrolidone/dialkylaminoalkyl acrylate or methacrylate copolymers, such as the products sold under the name “Gafquat®” by ISP, such as, for example, “Gafquat® 734” or “Gafquat® 755”, or alternatively the products known as “Copolymer® 845, 958 and 937”. These polymers are described in detail, for example, in French Patent Nos. FR 2 077 143 and 2 393 573,

fatty-chain polymers comprising a vinylpyrrolidone unit, such as the products sold under the name Styleze W20 and Styleze W10 by ISP,

dimethylaminoethyl methacrylate/vinylcaprolactam/vinylpyrrolidone terpolymers, such as the product sold under the name Gaffix VC 713 by ISP, and

quaternized vinylpyrrolidone/dimethylaminopropylmethacrylamide copolymers, such as the products sold under the name “Gafquate HS 100” by ISP;

(2) cationic polysaccharides, for example, comprising quaternary ammonium, such as those described in U.S. Pat. Nos. 3,589,578 and 4,031,307, such as guar gums comprising trialkylammonium cationic groups. Such products are sold, for example, under the trade names Jaguar C13 S, Jaguar C 15 and Jaguar C 17 by Meyhall;

(3) quaternary copolymers of vinylpyrrolidone and of vinylimidazole;

(4) chitosans or salts thereof; the salts that can be used are, for example, chitosan acetate, lactate, glutamate, gluconate or pyrrolidonecarboxylate.

Among these compounds, non-limiting mention may be made of chitosan having a degree of deacetylation of 90.5% by weight, sold under the name Kytan Brut Standard by Aber Technologies, and chitosan pyrrolidonecarboxylate sold under the name Kytamer® PC by Amerchol;

(5) cationic cellulose derivatives such as copolymers of cellulose or of cellulose derivatives grafted with a water-soluble monomer comprising a quaternary ammonium, and disclosed, for example, in U.S. Pat. No. 4,131,576, such as hydroxyalkylcelluloses, for instance hydroxymethyl-, hydroxyethyl- or hydroxypropylcelluloses grafted, for example, with a methacryloyloxyethyltrimethylammonium, methacrylam idopropyltrimethylammonium or dimethyldiallylammonium salt.

The products sold corresponding to this definition are, for example, the products sold under the name “Celquat L 200” and “Celquat H 100” by National Starch.

The amphoteric fixative polymers may be chosen from polymers comprising units B and C distributed randomly in the polymer chain, wherein B is chosen from a unit derived from a monomer comprising at least one basic nitrogen atom and C is chosen from a unit derived from an acid monomer comprising at least one carboxylic or sulphonic groups, or alternatively B and C can be chosen from groups derived from carboxybetaine or sulphobetaine zwitterionic monomers;

B and C may also be chosen from cationic polymer chains comprising primary, secondary, tertiary or quaternary amine groups, wherein at least one of the amine groups bears a carboxylic or sulphonic group connected via a hydrocarbon group or alternatively B and C form part of a chain of a polymer comprising an α,β-dicarboxylic ethylene unit wherein one of the carboxylic groups has been made to react with a polyamine comprising at least one primary or secondary amine groups.

The amphoteric fixative polymers corresponding to the definition given above may be chosen from:

(1) copolymers having acidic vinyl and basic vinyl units, such as those resulting from the copolymerization of a monomer derived from a vinyl compound bearing a carboxylic group such as, for example, acrylic acid, methacrylic acid, maleic acid, a-chloroacrylic acid, and a basic monomer derived from a substituted vinyl compound comprising at least one basic atom, such as dialkylaminoalkyl methacrylate and acrylate, dialkylaminoalkylmethacrylamides and -acrylamides. Such compounds are described in U.S. Pat. No. 3,836,537;

(2) polymers comprising units derived from:

a) at least one monomer chosen from acrylamides and methacrylamides substituted on the nitrogen atom by an alkyl group,

b) at least one acidic comonomer comprising at least one reactive carboxylic groups, and

c) at least one basic comonomer such as esters comprising primary, secondary, tertiary and quaternary amine substituents of acrylic and methacrylic acids and the product of quaternization of dimethylaminoethyl methacrylate with dimethyl or diethyl sulphate.

The N-substituted acrylamides or methacrylamides that may be used are chosen from compounds wherein the alkyl groups contain from 2 to 12 carbon atoms, for example, N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers may be chosen from acrylic acid, methacrylic acid, crotonic acid, itaconic acid, maleic acid and fumaric acid and alkyl monoesters, having 1 to 4 carbon atoms, of maleic or fumaric acids or anhydrides.

The basic comonomers may be chosen from aminoethyl, butylaminoethyl, N,N′-dimethylaminoethyl and N-tert-butylaminoethyl methacrylates.

In one embodiment, the copolymers whose CTFA (4th edition, 1991) name is Octylacrylamide/acrylates/butylaminoethyl methacrylate copolymer, such as the products sold under the name Amphomer® or Lovocryl® 47 by National Starch may be used;

(3) crosslinked and acylated polyaminoamides partially or totally derived from polyaminoamides of general formula: 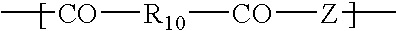
wherein
R₁₀ is chosen from divalent groups derived from a saturated dicarboxylic acids, mono- or dicarboxylic aliphatic acids comprising ethylenic double bonds, esters of a lower alkanol having 1 to 6 carbon atoms of these acids, and a group derived from the addition of any one of said acids with a bis(primary) or bis(secondary) amine, and
Z is chosen from a group deriving from a bis(primary), mono- or bis(secondary) polyalkylene-polyamine and may be chosen from:

a) in proportions ranging from 60 to 100 mol %, the group (IV):

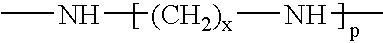

wherein x is 2 and p is chosen from 2 or 3, or alternatively x is 3 and p is 2

this group being derived from diethylenetriamine, from triethylenetetraamine or from dipropylenetriamine;

b) in proportions ranging from 0 to 40 mol %, the group (IV) above wherein x is 2 and p is 1 and which is derived from ethylenediamine, or the group deriving from piperazine:

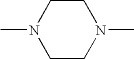

c) in proportions ranging from 0 to 20 mol %, the group —NH(CH₂)₆—NH— deriving from hexamethylenediamine,

these polyaminoamides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyaminoamide, and being acylated by the action of acrylic acid, chloroacetic acid or an alkane sultone, or salts thereof.

The saturated carboxylic acids may be chosen from acids having 6 to 10 carbon atoms, such as adipic acid, 2,2,4-trimethyladipic acid and 2,4,4-trimethyladipic acid, terephthalic acid, acids comprising an ethylenic double bond such as, for example, acrylic acid, methacrylic acid and itaconic acid.

The alkane sultones used in the acylation may be, for example, propane sultone or butane sultone; the salts of the acylating agents may be chosen from the sodium and potassium salts;

(4) polymers comprising zwitterionic units of formula: 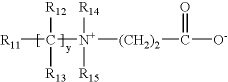
wherein:
R₁₁ is chosen from a polymerizable unsaturated group such as an acrylate, methacrylate, acrylamide or methacrylamide group,
y and z, which may be identical or different, are each chosen from a number ranging from 1 to 3,
R₁₂ and R₁₃, which may be identical or different, are each chosen from hydrogen atoms, methyl, ethyl and propyl groups,
R₁₄ and R₁₅, which may be identical or different, are each chosen from hydrogen atoms and alkyl groups such that the sum of the carbon atoms in R₁₄ and R₁₅ does not exceed 10.

The polymers comprising such units can also comprise units derived from non-zwitterionic monomers such as dimethyl- or diethylaminoethyl acrylate or methacrylate or alkyl acrylates or methacrylates, acrylamides or methacrylamides or vinyl acetate.

By way of example, mention may be made of the copolymers of methyl methacrylate/methyl dimethylcarboxymethylammonioethyl methacrylate such as the product sold under the name Diaformer Z301 by Sandoz;

(5) polymers derived from chitosan comprising monomer units corresponding to the following formulae: 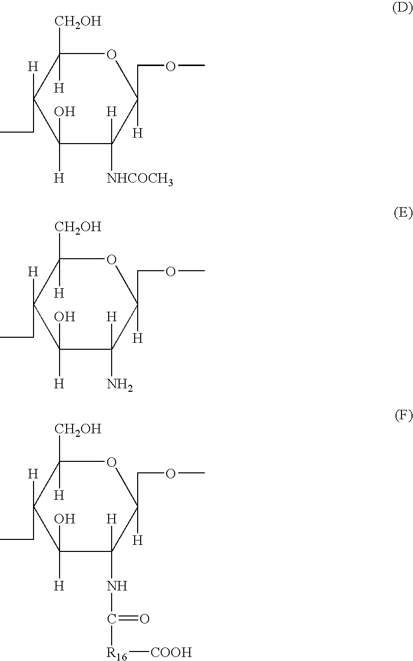
wherein:
the unit (D) is present in an amount ranging from 0 to 30%,
the unit (E) is present in an amount ranging from 5% to 50% and
the unit (F) is present in an amount ranging from 30% to 90%, wherein R₁₆ is chosen from: 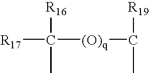
   wherein,
if q is 0, R₁₇, R₁₈ and R₁₉, which may be identical or different, are each chosen from hydrogen atoms, methyl, hydroxyl, acetoxy or amino residues, monoalkylamine residues and dialkylamine residues that are optionally interrupted by at least one nitrogen atom and/or optionally substituted by at least one amine, hydroxyl, carboxyl, alkylthio or sulphonic group, an alkylthio residue wherein the alkyl group bears an amino residue, at least one of the groups R₁₇, R₁₈ and R₁₉ are, in this case, a hydrogen atom;

or, if q is 1, R₁₇, R₁₈ and R₁₉ are each a hydrogen atom, as well as the salts formed by these compounds with bases or acids.

(6) Polymers corresponding to the formula below are described, for example, in French Patent No. FR 1 400 366: 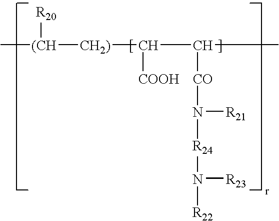
wherein:
R₂₀ is chosen from hydrogen atoms, CH₃O, CH₃CH₂O groups and phenyl groups,
R₂₁ is chosen from hydrogen atoms and lower alkyl groups such as methyl or ethyl,
R₂₂ is chosen from hydrogen atoms and C₁-C₆ lower alkyl groups such as methyl or ethyl,
R₂₃ is chosen from C₁-C₆ lower alkyl groups such as methyl or ethyl and groups corresponding to the formula: —R₂₄—N(R₂₂)₂,
R₂₄ is chosen from —CH₂—CH₂—, —CH₂—CH₂—CH₂— and —CH₂—CH(CH₃)— groups;

(7) polymers derived from the N-carboxyalkylation of chitosan, such as N-carboxymethylchitosan or N-carboxybutylchitosan sold under the name “Evalsan” by Jan Dekker;

(8) amphoteric polymers of the type -D-X-D-X chosen from:
a) polymers obtained by the action of chloroacetic acid or sodium chloroacetate on compounds comprising at least one unit of formula:
   -D-X-D-X-D- (VI)
   wherein D is chosen from a group 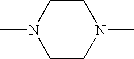
   and X is chosen from the symbol E or E′,
   wherein:
E or E′, which may be identical or different, are each chosen from divalent groups that are alkylene groups with straight or branched chains comprising up to 7 carbon atoms in the main chain, which is unsubstituted or substituted by hydroxyl groups and which can comprise, in addition to the oxygen, nitrogen and sulphur atoms, 1 to 3 aromatic and/or heterocyclic rings; the oxygen, nitrogen and sulphur atoms being present in the form of ether, thioether, sulphoxide, sulphone, sulphonium, alkylamine or alkenylamine groups, hydroxyl, benzylamine, amine oxide, quaternary ammonium, amide, imide, alcohol, ester and/or urethane groups;
   b) polymers of formula:
      -D-X-D-X- (VI′)

where D is chosen from a group 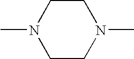
and X is chosen from the symbol E or E′ and at least once E′; E having the meaning given above and E′ is chosen from divalent groups that are alkylene groups with a straight or branched chain having up to 7 carbon atoms in the main chain, which is unsubstituted or substituted by at least one hydroxyl group and comprises at least one nitrogen atom, the nitrogen atom being substituted by an alkyl chain that is optionally interrupted by an oxygen atom and necessarily comprising at least one carboxyl function or at least one hydroxyl function and betainized by reaction with chloroacetic acid or sodium chloroacetate;

(9) (C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers can also comprise other vinyl comonomers such as vinylcaprolactam.

The amphoteric fixative polymers may, for example, be chosen from those of class (3), such as the copolymers whose CTFA name is Octylacrylamide/acrylates/butylaminoethyl methacrylate copolymer, such as the products sold under the names Amphomer®, Amphomer® LV 71 or Lovocryl® 47 by National Starch and those of class (4) such as the copolymers of methyl methacrylate/methyl dimethylcarboxymethylammonioethyl methacrylate, sold, for example, under the name Diaformer Z301 by Sandoz.

The nonionic fixative polymers may be chosen, for example, from:

polyalkyloxazolines;

vinyl acetate homopolymers;

vinyl acetate copolymers, for instance copolymers of vinyl acetate and of acrylic ester; copolymers of vinyl acetate and of ethylene, or copolymers of vinyl acetate and of maleic ester, for example of dibutyl maleate;

acrylic ester homopolymers and copolymers, for instance copolymers of alkyl acrylates and of alkyl methacrylates, such as the products sold by Rohm & Haas under the names Primal® AC-261 K and Eudragit® NE 30 D, by BASF under the name 8845, or by Hoechst under the name Appretan® N9212;

acrylonitrile copolymers and copolymers of a nonionic monomer selected, for example, from butadiene and alkyl (meth)acrylates; non-limiting mention may be made of the products sold under the name CJ 0601 B by Rohm & Haas;

styrene homopolymers;

styrene copolymers, for instance copolymers of styrene and of an alkyl (meth)acrylate, such as the products Mowilith® LDM 6911, Mowilith® DM 611 and Mowilith® LDM 6070 sold by Hoechst, and the products Rhodopas® SD 215 and Rhodopas® DS 910 sold by Rhone-Poulenc; copolymers of styrene, of alkyl methacrylate and of alkyl acrylate; copolymers of styrene and of butadiene; or copolymers of styrene, of butadiene and of vinylpyridine;

polyamides;

vinyllactam homopolymers other than vinylpyrrolidone homopolymers, such as the polyvinylcaprolactam sold under the name Luviskol® Plus by BASF; and

vinyllactam copolymers such as a poly(vinylpyrrolidone/vinyllactam) copolymer sold under the trade name Luvitec® VPC 55K65W by BASF, poly(vinylpyrrolidone/vinyl acetate) copolymers, such as those sold under the name PVPVA® S630L by ISP, Luviskol® VA 73, VA 64, VA 55, VA 37 and VA 28 by BASF; and poly(vinylpyrrolidone/vinyl acetate/vinyl propionate) terpolymers, for instance the product sold under the name Luviskol® VAP 343 by BASF.

The alkyl groups of the nonionic polymers mentioned above may contain from 1 to 6 carbon atoms.

According to one embodiment, it is also possible to use fixative polymers of grafted silicone type comprising a polysiloxane portion and a portion consisting of a non-silicone organic chain, one of the two portions constituting the main chain of the polymer and the other being grafted onto said main chain.

These polymers are described, for example, in European Patent Application Nos. EP-A-0 412 704, EP-A-0 412 707, EP-A-0 640 105 and WO 95/00578, EP-A-0 582 152 and WO 93/23009 and U.S. Pat. Nos. 4,693,935, 4,728,571 and 4,972,037.

These polymers may be amphoteric, anionic or nonionic, for example, anionic or nonionic.

Such polymers are, for example, copolymers that can be obtained by free radical polymerization from the monomer mixture formed from:

a) 50% to 90% by weight of tert-butyl acrylate;

b) 0 to 40% by weight of acrylic acid;

c) 5% to 40% by weight of a silicone macromer of formula: 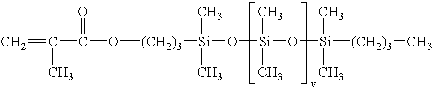
wherein v is a number ranging from 5 to 700, the weight percentages being calculated relative to the total weight of the monomers.

Other examples of grafted silicone polymers are, for example, polydimethylsiloxanes (PDMSs) onto which are grafted, via a thiopropylene-type connecting chain, mixed polymer units of the poly(meth)acrylic acid type and of the polyalkyl (meth)acrylate type and polydimethylsiloxanes (PDMSS) onto which are grafted, via a thiopropylene-type connecting chain, polymer units of the polyisobutyl (meth)acrylate type.

Another type of silicone fixative polymer that may be mentioned is the product Luviflex® Silk, sold by BASF.

Functionalized or non-functionalized, silicone or non-silicone, cationic, nonionic, anionic or amphoteric polyurethanes or mixtures thereof may also be used as fixative polymers.

The polyurethanes may be chose from those disclosed in European Application Nos. EP 0 751 162, EP 0 637 600, EP 0 656 021, EP 0 619 111, and EP 0 648 485 and French Patent Application No. FR 2 743 297, and also published PCT Application WO 94/03510.

The polyurethanes which may be used include products sold under the names Luviset Pur® and Luviset® Si Pur by BASF.

The concentration of fixative polymer(s) used in the compositions disclosed herein ranges from 0.1% to 20%, for example, from 0.5% to 10% by weight, relative to the total weight of the composition.

Thickners

Natural thickeners suitable for the disclosure include, for example, xanthan gum, scleroglucan gum, gellan gum, rhamsan gum, alginates, maltodextrin, starch and its derivatives, karaya gum, carob flour and guar gums.

The synthetic thickeners may be chosen from polymers or copolymers of acrylic and/or methacrylic acid, such as acrylic acid/ethyl acrylate copolymers and carboxyvinyl polymers. Examples of such polymers or copolymers are, for example, the “carbomers” (CTFA) sold by Goodrich under the name Carbopol or the polyglyceryl methacrylate sold by Guardian under the name Lubragel, or else the polyglyceryl acrylate sold under the name Hispagel by Hispano Chimica.

Other acrylic compounds include copolymers of acrylic or methacrylic acid comprising at least one C1 to C30 alkyl acrylate unit and/or one urethane unit optionally substituted by a fatty chain. Mention may be made, for example, of Pemulen TR1 (Goodrich), Viscophobe DB 1000 (Union Carbide) and Acrysol 44 or 46 (Rohm & Haas).

As thickeners it is also possible to use polyethylene glycols (PEG) and derivatives thereof.

It is also possible to use as thickener, thickening polyacrylamides. These may be chosen from:

crosslinked 2-acrylamido-2-methylpropanesulphonic homopolymers,

optionally crosslinked acrylamide-ammonium acrylate copolymers,

optionally crosslinked acrylamide (or methacrylamide)-methacryloyloxyethyltrimethylammonium chloride copolymers,

optionally crosslinked, fully or partly neutralized acrylamide-2-acrylamido-2-methylpropanesulphonic acid copolymers.

The crosslinked acrylamide/ammonium acrylate copolymers may be chosen from acrylamide/ammonium acrylate (5/95 by weight) copolymers crosslinked with a crosslinking agent comprising olefinic polyunsaturation, such as divinylbenzene, tetraallyloxyethane, methylenebisacrylamide, diallyl ether, polyallyl polyglyceryl ethers or allyl ethers of alcohol of the sugar series, such as erythritol, pentaerythritol, arabitol, mannitol, sorbitol or glucose.

Similar copolymers are described and prepared in French Patent No. FR-2 416 723 and in U.S. Pat. Nos. 2,798,053 and 2,923,692.

This type of crosslinked copolymer may be used, for example, in the form of a water-in-oil emulsion composed of 30% by weight of said copolymer, 25% by weight of liquid paraffin, 4% by weight of a mixture of sorbitan stearate and a hydrophilic ethoxylated derivative, and 41% by weight of water. An emulsion of this kind is sold under the name “Bozepol C” by Hoechst.

The acrylamide-2-acrylamido-2-methylpropanesulphonic acid copolymers may be chosen from copolymers which are crosslinked by a compound comprising olefinic polyunsaturation, such as those mentioned above, and are fully or partly neutralized by a neutralizing agent such as sodium hydroxide, potassium hydroxide, aqueous ammonia or an amine such as triethanolamine or monoethanolamine.

They may be prepared by copolymerizing acrylamide and sodium 2-acrylamido-2-methylpropanesulphonate by a free-radical route using initiators of azobisisobutyronitrile type and by precipitation from an alcohol such as tert-butanol.

Copolymers may be chosen from those obtained by copolymerizing 70 to 55 mol % of acrylamide and 30 to 45 mol % of sodium 2-acrylamido-2-methylpropanesulphonate. The crosslinking agent is used at concentrations of 10⁻⁴ to 4×10⁻⁴ mol per mole of the monomer mixture.

These copolymers may be incorporated into the compositions disclosed herein in the form of water-in-oil emulsions comprising from 35% to 40% by weight of this copolymer, from 15% to 25% by weight of a mixture of C₁₂-C₁₃ isoparaffinic hydrocarbons, from 3% to 8% by weight of polyethylene glycol lauryl ether comprising 7 mol of ethylene oxide, and water. An emulsion of this kind is sold under the name “Sepigel 305” by SEPPIC.

The crosslinked acrylamide-methacryloyloxyethyltrimethylammonium chloride copolymer may be chosen from a copolymer obtained by copolymerizing acrylamide and dimethylaminoethyl methacrylate quaternized with methyl chloride, followed by crosslinking with an olefinically unsaturated compound, for example, methylenebisacrylamide.

The copolymer may also be chosen from a crosslinked acrylamide/methacryloyloxyethyltrimethylammonium chloride (approximately 50/50 by weight) copolymer in the form of a dispersion comprising 50% by weight of said copolymer in mineral oil. This dispersion is sold under the name “Salcare SC92” by Allied Colloids.

The non-crosslinked methacrylamide-methacryloyloxyethyltrimethylammonium chloride copolymers are, for example, the products sold under the trade names Rohagit KF400 and KF720 by Rohm & Haas.

Possible Surfactants

The surfactants may be chosen from:

(i) Anionic Surfactant(s):

In the context of the present disclosure, their nature does not represent a truly critical factor.

The anionic surfactants may be chosen from, alone or as mixtures, of salts (for example, alkali metal salts such as sodium salts, ammonium salts, amine salts, amino alcohol salts or alkaline earth metal (magnesium) salts) of the following compounds: alkyl sulphates, alkyl ether sulphates, alkylamido ether sulphates, alkylaryl polyether sulphates, monoglyceride sulphates; alkylsulphonates, alkyl phosphates, alkylamidesulphonates, alkylarylsulphonates, α-olefinsulphonates, paraffinsulphonates; alkylsulphosuccinates, alkyl ether sulphosuccinates, alkylamide-sulphosuccinates; alkylsulphosuccinamates; alkyl sulphoacetates; alkyl ether phosphates; acylsarcosinates; acylisethionates and N-acyltaurates, the alkyl or acyl radical of all of these various compounds may, for example, comprise from 12 to 20 carbon atoms, and the aryl radical may be chosen from phenyl groups and benzyl groups. Among the anionic surfactants which can also be used, non-limiting mention may also be made of fatty acid salts such as the salts of oleic, ricinoleic, palmitic and stearic acids, coconut oil acid or hydrogenated coconut oil acid; acyl lactylates wherein the acyl radical contains 8 to 20 carbon atoms. Weakly anionic surfactants may also be used, such as alkyl-D-galactoside uronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof.

The anionic surfactants may be chosen, for example, from alkyl sulphate salts and alkyl ether sulphate salts and mixtures thereof.

(ii) Nonionic Surfactant(s):

The nonionic surfactants are, also, compounds that are well known per se (see, for example, in this respect “Handbook of Surfactants” by M. R. Porter, published by Blackie & Son (Glasgow and London), 1991, pp. 116-178) and, in the context of the present disclosure, their nature is not a critical feature. Thus, they may be chosen from (non-limiting list) polyethoxylated, polypropoxylated or polyglycerolated fatty acids, alkylphenols, x-diols or alcohols having a fatty chain comprising, for example, 8 to 18 carbon atoms, it being possible for the number of ethylene oxide or propylene oxide groups to range, for example, from 2 to 50 and for the number of glycerol groups to range, for example, from 2 to 30. Non-limiting mention may also be made of copolymers of ethylene oxide and of propylene oxide, condensates of ethylene oxide and of propylene oxide with fatty alcohols; polyethoxylated fatty amides, for example, having from 2 to 30 mol of ethylene oxide, polyglycerolated fatty amides comprising on average 1 to 5, for example, 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

(iii) Amphoteric Surfactant(s):

The amphoteric surfactants, whose nature is not a critical feature in the context of the present disclosure, may be chosen from aliphatic secondary or tertiary amine derivatives wherein the aliphatic radical is a linear or branched chain comprising 8 to 22 carbon atoms and comprising at least one water-soluble anionic group (for example carboxylate, sulphonate, sulphate, phosphate or phosphonate); non-limiting mention may also be made of (C8-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the amine derivatives, mention may be made of the products sold under the name Miranol®, as described in U.S. Pat. Nos. 528,378 and 2,781,354 and having the structures:
R₂—CONHCH₂CH₂—N(R₃)(R₄)(CH₂COO—) (2)
wherein:
R₂ is chosen from alkyl radicals derived from an acid R₂—COOH present in hydrolysed coconut oil, heptyl, nonyl or undecyl radicals,
R₃ is chosen from β-hydroxyethyl groups and
R₄ is chosen from carboxymethyl groups; and
   R_{2′}—CONHCH₂CH₂—N(B)(C) (3)
   wherein:

B is chosen from —CH₂CH₂OX′,

C is chosen from —(CH₂)_{z}—Y′, wherein z is 1 or 2,

X′ is chosen from —CH₂CH₂—COOH groups or hydrogen atoms,

Y′ is chosen from —COOH or —CH₂—CHOH—SO₃H radicals,

R₂ is chosen from alkyl radicals of an acid R₉—COOH present in coconut oil or in hydrolyzed linseed oil, alkyl radicals, for example C₇, C₉, C₁₁ or C₁₃ alkyl radicals, C₁₇ alkyl radicals and their iso form, or unsaturated C₁₇ radicals.

These compounds are classified in the CTFA dictionary, 5th edition, 1993, under the names Disodium Cocoamphodiacetate, Disodium Lauroamphodiacetate, Disodium Caprylamphodiacetate, Disodium Capryloamphodiacetate, Disodium Cocoamphodipropionate, Disodium Lauroamphodipropionate, Disodium Caprylamphodipropionate, Disodium Capryloamphodipropionate, Lauroamphodipropionic acid, Cocoamphodipropionic acid.

By way of example, mention may be made of the cocoamphodiacetate sold under the trade name Miranol® C2M concentrate by Rhodia.

In the compositions disclosed herein, mixtures of surfactants may be used, for example, mixtures of anionic surfactants and of amphoteric or nonionic surfactants. One mixture that may be used is a mixture consisting of at least one anionic or nonionic surfactant and of at least one amphoteric surfactant.

The anionic surfactant may be chosen from sodium, triethanolamine or ammonium (C₁₂-C₁₄)alkyl sulphates, sodium, triethanolamine or ammonium (C₁₂-C₁₄)alkyl ether sulphates oxyethylenated with 2.2 mol of ethylene oxide, sodium cocoyl-isethionate and sodium α-(C₁₄-C₁₆)olefinsulphonate, and mixtures thereof, with:

either an amphoteric surfactant such as the amine derivatives known as disodium cocoamphodipropionate or sodium cocoamphopropionate sold, for example, by Rhodia under the trade name Miranol® C2M CONC as an aqueous solution comprising 38% active substance, or under the name Miranol® C32;

or an amphoteric surfactant of zwitterionic type such as alkylbetaines, for example, the cocoylbetaine sold under the name Dehyton® AB 30 as an aqueous solution comprising 32% a.s. by Henkel or alkylamidobetaines such as Tegobetaine® F50 sold by Goldschmidt.

Cationic Surfactants

The composition disclosed herein comprises at least one cationic surfactants, such as optionally polyoxyalkylenated primary, secondary or tertiary fatty amine salts, quaternary ammonium salts, and mixtures thereof.

Examples of quaternary ammonium salts include:

those of general formula (VI) below:

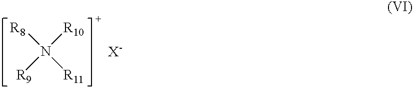

wherein
R₈ to R₁₁, which may be identical or different, are each chosen from linear or branched aliphatic radicals comprising from 1 to 30 carbon atoms, and aromatic radicals such as aryl or alkylaryl. The aliphatic radicals can comprise heteroatoms such as, for example, oxygen, nitrogen, sulphur or halogens. The aliphatic radicals are selected, for example, from alkyl, alkoxy, polyoxy(C₂-C₆)alkylene, alkylamide, (C₁₂-C₂₂)alkylamido(C₂-C₆)alkyl, (C₁₂-C₂₂)alkyl acetate and hydroxyalkyl radicals, comprising from 1 to 30 carbon atoms;
X is chosen from halides, phosphates, acetates, lactates, (C₂-C₆)alkyl sulphates and alkyl- or alkylaryl-sulphonates;

quaternary ammonium salts of imidazoline, such as, for example, those of formula (VIl) below:

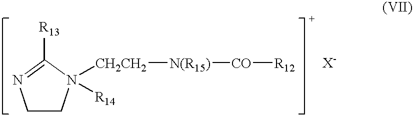

wherein:
R₁₂ is chosen from alkenyl or alkyl radicals comprising from 8 to 30 carbon atoms, for example tallow fatty acid derivatives,
R₁₃ is chosen from hydrogen atoms, C₁-C₄ alkyl radicals and alkenyl or alkyl radicals comprising from 8 to 30 carbon atoms,
R₁₄ is chosen from C₁-C₄ alkyl radicals,
R₁₅ is chosen from hydrogen atoms and C₁-C₄ alkyl radicals,
X⁻ is chosen from halides, phosphates, acetates, lactates, alkyl sulphates and alkyl- or alkylaryl-sulphonates.
R₁₂ and R₁₃ may be chosen from, for example, a mixture of alkenyl or alkyl radicals comprising from 12 to 21 carbon atoms, for example tallow fatty acid derivatives, R₁₄ may be chosen from a methyl radical and R₁₅ may be chosen from a hydrogen atom. A product of this kind is sold, for example, under the name Rewoquat® W 75 by Rewo;

diquaternary ammonium salts of formula (VIII):

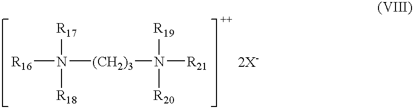

wherein:
R₁₆ is chosen from aliphatic radicals comprising from 16 to 30 carbon atoms,
R₁₇, R₁₈, R₁₉, R₂₀ and R₂₁, which may be identical or different, are each chosen from hydrogen and alkyl radicals comprising from 1 to 4 carbon atoms, and
X is chosen from halides, acetates, phosphates, nitrates and methyl sulphates. Such diquaternary ammonium salts may, for example, comprise propane tallow diammonium dichloride;

quaternary ammonium salts comprising at least one ester function, such as those of formula (IX) below: 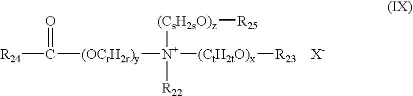
wherein:

R₂₂ is chosen from C₁-C₆ alkyl radicals and C₁-C₆ hydroxyalkyl or dihydroxyalkyl radicals;

R₂₃ is chosen from:

a radical

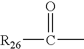

linear or branched, saturated or unsaturated C₁-C₂₂ hydrocarbon radicals R₂₇,

a hydrogen atom,

R₂₅ is chosen from:

a radical

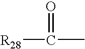

linear or branched, saturated or unsaturated C₁-C₆ hydrocarbon radicals R₂₉, and

a hydrogen atom,

R₂₄, R₂₆ and R₂₈, which may be identical or different, are each chosen from linear or branched, saturated or unsaturated C₇-C₂₁ hydrocarbon radicals;

r, s and t, which may be identical or different, are each chosen from integers ranging from 2 to 6;

y is chosen from an integer ranging from 1 to 10;

x and z, which may be identical or different, are each chosen from integers ranging from 0 to 10;

X is chosen from simple or complex, organic or inorganic anions;

with the proviso that the sum x+y+z is from 1 to 15, that when x is 0, then R₂₃ is chosen from R₂₇ and that when z is 0, then R₂₅ is chosen from R₂₉.

The R₂₂ alkyl radicals may be linear or branched, for example, linear.

R₂₂ may be, for example, chosen from methyl, ethyl, hydroxyethyl and dihydroxypropyl radicals, for example, methyl or ethyl radicals.

The sum x+y+z may be, for example, from 1 to 10.

When R₂₃ is a hydrocarbon radical R₂₇, it may be long and contain from 12 to 22 carbon atoms, or short and contain from 1 to 3 carbon atoms.

When R₂₅ is a hydrocarbon radical R₂₉, it may, for example, contain from 1 to 3 carbon atoms.

R₂₄, R₂₆ and R₂₈, which may be identical or different, are each chosen from, for example, linear or branched, saturated or unsaturated C₁₁-C₂, hydrocarbon radicals, for example, from linear or branched, saturated or unsaturated, C₁₁-C₂₁ alkyl and alkenyl radicals.

x and z, which may be identical or different, are each chosen, for example, from 0 and 1.

y may be, for example, 1.

r, s and t, which may be identical or different, may be, for example, 2 or 3, for example, 2.

The anion may be chosen from halides (chloride, bromide or iodide) and alkyl sulphates, for example, methyl sulphate. However, methanesulphonate, phosphate, nitrate, tosylate, an anion derived from an organic acid, such as acetate or lactate, or any other anion that is compatible with the ester-functional ammonium may be used.

The anion X⁻ may also, for example, be chosen from chloride and methyl sulphate.

The ammonium salts may be chosen from those of formula (IX) wherein:

R₂₂ is chosen from a methyl or ethyl radical,

x and y are equal to 1;

z is chosen from 0 and 1;

r, s and t are equal to 2;

R₂₃ is chosen from:
a radical 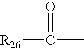
methyl, ethyl or C₁₄-C₂₂ hydrocarbon radicals;
a hydrogen atom;

R₂₅ is chosen from:
a radical 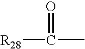
a hydrogen atom;

R₂₄, R₂₆ and R₂₈, which may be identical or different, are each chosen from linear or branched, saturated or unsaturated C₁₃-C₁₇ hydrocarbon radicals, for example, from linear or branched, saturated or unsaturated C₁₃-C₁₇ alkyl and alkenyl radicals.

The hydrocarbon radicals may, for example, be linear.

Examples that may be mentioned include the compounds of formula (IX) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulphate, for example), and mixtures thereof. The acyl radicals may, for example, contain 14 to 18 carbon atoms and are obtained, for example, from a plant oil such as palm oil or sunflower oil. When the compound comprises at least two acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization using an alkylating agent such as an alkyl halide (for example, a methyl or ethyl halide), a dialkyl sulphate (for example, dimethyl or diethyl sulphate), methyl methanesulphonate, methyl para-toluenesulphonate, glycol chlorohydrin or glycerol chlorohydrin.

Such compounds are sold, for example, under the names Dehyquart® by Henkel, Stepanquat® by Stepan, Noxamium® by CECA or Rewoquat® WE 18 by Rewo-Witco.

The composition disclosed herein may comprise a mixture of quaternary ammonium mono-, di- and triester salts with a majority by weight of diester salts.

As a mixture of ammonium salts it is possible for example to use the mixture comprising 15% to 30% by weight of acyloxyethyl-dihydroxyethyl-methylammonium methyl sulphate, 45% to 60% of diacyloxyethyl-hydroxyethyl-methylammonium methyl sulphate and 15% to 30% of triacyloxyethyl-methylammonium methyl sulphate, the acyl radicals having 14 to 18 carbon atoms and originating from optionally partially hydrogenated palm oil.

It is also possible to use the ammonium salts comprising at least one ester function that are described in U.S. Pat. No. 4,874,554 and U.S. Pat. No. 4,137,180.

The quaternary ammonium salts of formula (VI) may be chosen from—

-tetraalkylammonium chlorides such as, for example, dialkyldimethylammonium chlorides and alkyltrimethylammonium chlorides, wherein the alkyl radical contains from about 12 to 22 carbon atoms, for example, behenyltrimethylammonium chloride, distearyidimethylammonium chloride, cetyltrimethylammonium chloride, or benzyldimethylstearylammonium chloride, and

-palmitylamidopropyltrimethylammonium chloride or stearamidopropyldimethyl(myristyl acetate)ammonium chloride sold under the name Ceraphyle 70 by Van Dyk.

The cationic surfactants may be chosen from quaternary ammonium salts, for example, behenyltrimethylammonium chloride and palmitylamidopropyltrimethylammonium chloride.

The composition disclosed herein may comprise the surfactant or surfactants in an amount ranging from 0.1% to 10% by weight, for example, from 0.5% to 8% by weight and further, for example, from 1% to 5% by weight, relative to the total weight of the composition.

The surfactant selected may be, for example, an amphoteric or nonionic surfactant. The skilled person will ensure that any additives and their amount are selected such that they are not detrimental to the properties of the compositions according to the present disclosure.

These additives are present in the composition according to the disclosure in an amount ranging from 0 to 20% by weight, relative to the total weight of the composition.

The aerosol devices of the present disclosure may be provided in the form of mousses or hair lacquers.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLE

We prepared a lacquer according to the disclosure:

| | | |
|---|---|---|
| a. Avalure UR 450 (Noveon) | 2% | a.s. |
| b. water | 58% | a.s. |
| c. DME | 40% | a.s. |

We prepared another lacquer according to the disclosure:

| | | |
|---|---|---|
| a. Avalure UR 450 (Noveon) | 3% | a.s. |
| b. Ethanol | 45% | a.s. |
| c. water | 17% | a.s. |
| d. DME | 35% | a.s. |

We prepared a mousse according to the disclosure:

| | | |
|---|---|---|
| a. Avalure UR 450 (Noveon) | 1.8% | a.s. |
| b. oxyethylenated (20EO) sorbitan monolaurate | 0.2% | a.s. |
| c. water | 93% | a.s. |
| d. isobutane/propane/butane (50/25/25) | 5% | a.s. |

We prepared a water/air-type fixative spray in a container under pressure according to the disclosure. The fluid comprised:

| | | |
|---|---|---|
| a. Avalure UR 450 (Noveon) | 3% | a.s. |
| b. water | 97% | a.s. |

The compositions obtained were easy to apply and gave the hair, after drying, excellent holding properties that resisted water. The hair also had good cosmetic properties.

What is claimed is:

1. A composition for cosmetic treatment of keratin materials, packaged in an aerosol device comprising (a) at least one propellant and (b) a hairstyling composition comprising at least one polyurethane having a number-average molecular weight ranging from 400 000 to 5 000 000 g/mol, in a cosmetically acceptable medium comprising water.

2. A composition for cosmetic treatment of keratin materials according to claim 1, wherein the at least one polyurethane has a molecular mass ranging from 400 000 to 3 000 000.

3. A composition for cosmetic treatment of keratin materials according to claim 2, wherein the at least one polyurethane has a molecular mass ranging from 1000 000 to 2 500 000.

4. A composition for cosmetic treatment of keratin materials according to claim 1, wherein the at least one polyurethane is nonassociative.

5. A composition for cosmetic treatment of keratin materials according to claim 1, wherein the at least one polyurethane comprises a base repeating unit conforming to the general formula (I):
—O—B—O—CO—NH—R—NH—CO— (I)
wherein:
B is chosen from C₁ to C₃₀ divalent hydrocarbon groups, which are unsubstituted or substituted by a group chosen from at least one carboxylic acid function and at least one sulfonic acid function, wherein the at least one carboxylic acid function and the at least one sulfonic acid function are in free form or are fully or partially neutralized by an inorganic or organic base, and
R is chosen from divalent groups chosen from C₁ to C₂₀ aliphatic hydrocarbon groups, C₃ to C₂₀ cycloaliphatic hydrocarbon groups and C₆ to C₂₀ aromatic hydrocarbon groups, and combinations thereof, wherein each group is substituted or unsubstituted.

6. A composition for cosmetic treatment of keratin materials according to claim 4, wherein the at least one polyurethane is present in an amount ranging from 0.05% to 40% by weight, relative to the total weight of the composition.

7. A composition for cosmetic treatment of keratin materials according to claim 6, wherein the at least one polyurethane is present in an amount ranging from 0.10% to 20% by weight, relative to the total weight of the composition.

8. A composition for cosmetic treatment of keratin materials according to claim 1, wherein the cosmetically acceptable medium comprises water ranging from 50% to 95% by weight, relative to the total weight of the mixture comprised in the aerosol device.

9. A composition for cosmetic treatment of keratin materials according to claim 1, wherein the at least one propellant (a) is present in an amount ranging from 1% to 90% by weight, relative to the total weight of the mixture comprised in the aerosol device.

10. A composition for cosmetic treatment of keratin materials according to claim 9, wherein the at least one propellant is chosen from propane, n-butane, isobutane, DME, and mixtures thereof.

11. An aerosol device comprising a composition for cosmetic treatment of keratin materials, packaged in an aerosol device comprising (a) at least one propellant and (b) a hairstyling composition comprising at least one polyurethane having a number-average molecular weight ranging from 400 000 to 5 000 000 g/mol, in a cosmetically acceptable medium comprising water.

12. A method for styling the hair comprising spraying the mixture comprised in an aerosol device comprising a composition for cosmetic treatment of keratin materials, packaged in an aerosol device comprising (a) at least one propellant and (b) a hairstyling composition comprising at least one polyurethane having a number-average molecular weight ranging from 400 000 to 5 000 000 g/mol, in a cosmetically acceptable medium comprising water over the hair that has been shaped and in allowing the hair thus treated to dry.

13. A process for treating the hair comprising spraying to said hair the mixture comprised in an aerosol device comprising a composition for cosmetic treatment of keratin materials, packaged in an aerosol device comprising (a) at least one propellant and (b) a hairstyling composition comprising at least one polyurethane having a number-average molecular weight ranging from 400 000 to 5 000 000 g/mol, in a cosmetically acceptable medium comprising water wherein the hair is fixed and shaped and retains the shape of hairstyles when said hair comes into contact with water for a prolonged period.
